SEQUENCE LISTING (18 sequences)

SEQ ID NO: 1 - protein, 15 aa, synthetic construct
  SITE 8..9: note = Residues linked via 1,3 oxazinane
SGCGTKRTQQTKGWY

SEQ ID NO: 2 - protein, 14 aa, synthetic construct
  SITE 7..8: note = Residues linked via 1,3 oxazinane
GCGTKRTQQTKGWY

SEQ ID NO: 3 - protein, 17 aa, synthetic construct
  SITE 10..11: note = Residues linked via 1,3 oxazinane
TLSGCGTKRTQQTKGWY

SEQ ID NO: 4 - protein, 16 aa, synthetic construct
  SITE 9..10: note = Residues linked via 1,3 oxazinane
LSGCGTKRTQQTKGWY

SEQ ID NO: 5 - protein, 14 aa, synthetic construct
  SITE 8..9: note = Residues linked via 1,3 oxazinane
SGCGTKRTQQTKGW

SEQ ID NO: 6 - protein, 13 aa, synthetic construct
  SITE 7..8: note = Residues linked via 1,3 oxazinane
GCGTKRTQQTKGW

SEQ ID NO: 7 - protein, 15 aa, synthetic construct
  SITE 10..11: note = Residues linked via 1,3 oxazinane
TLSGCGTKRTQQTKG

SEQ ID NO: 8 - protein, 14 aa, synthetic construct
  SITE 9..10: note = Residues linked via 1,3 oxazinane
LSGCGTKRTQQTKG

SEQ ID NO: 9 - protein, 13 aa, synthetic construct
  SITE 8..9: note = Residues linked via 1,3 oxazinane
SGCGTKRTQQTKG

SEQ ID NO: 10 - protein, 12 aa, synthetic construct
  SITE 7..8: note = Residues linked via 1,3 oxazinane
GCGTKRTQQTKG

SEQ ID NO: 11 - protein, 10 aa, synthetic construct
  SITE 5..6: note = Residues linked via 1,3 oxazinane
GTKRTQQTKG

SEQ ID NO: 12 - protein, 16 aa, synthetic construct
  SITE 12..13: note = Residues linked via 1,3 oxazinane
IETLSGCGTKRTQQTK

SEQ ID NO: 13 - protein, 14 aa, synthetic construct
  SITE 10..11: note = Residues linked via 1,3 oxazinane
TLSGCGTKRTQQTK

SEQ ID NO: 14 - protein, 13 aa, synthetic construct
  SITE 9..10: note = Residues linked via 1,3 oxazinane
LSGCGTKRTQQTK

SEQ ID NO: 15 - protein, 12 aa, synthetic construct
  SITE 8..9: note = Residues linked via 1,3 oxazinane
SGCGTKRTQQTK

SEQ ID NO: 16 - protein, 11 aa, synthetic construct
  SITE 7..8: note = Residues linked via 1,3 oxazinane
GCGTKRTQQTK

SEQ ID NO: 17 - protein, 10 aa, synthetic construct
  SITE 6..7: note = Residues linked via 1,3 oxazinane
CGTKRTQQTK

SEQ ID NO: 18 - protein, 9 aa, synthetic construct
  SITE 5..6: note = Residues linked via 1,3 oxazinane
GTKRTQQTK

(19) United States
(12) Patent Application Publication
(10) Pub. No.: US 2025/0025527 A1
(43) Pub. Date: Jan. 23, 2025

(54) THREOGLUCINS AS POTENT-ANTIBACTERIAL AGENTS AGAINST STREPTOCOCCUS SUIS

(71) Applicant: The Trustees of Princeton University, Princeton, NJ (US)

(72) Inventors: Mohammad R. Seyedsayamdost, Princeton, NJ (US); Brett C. Covington, New Brunswick, NJ (US); Kenzie A. Clark, Princeton, NJ (US)

(73) Assignee: The Trustees of Princeton University, Princeton, NJ (US)

(21) Appl. No.: 18/777,763

(22) Filed: Jul. 19, 2024

Related U.S. Application Data
(60) Provisional application No. 63/527,633, filed on Jul. 19, 2023.

(51) Int. Cl. A61K 38/10; A61P 31/04

(52) U.S. Cl. CPC A61K 38/10; A61P 31/04

(57) ABSTRACT

Compositions are provided that can be used to, e.g., treat or prevent infections caused by Streptococcus suis. The compositions may include one or more threoglucins in a carrier, at a total concentration of threoglucins being at least 1 μM. The at least one threoglucin may include one or more of threoglucins A-R. Such threoglucins may be produced by, e.g., growing a culture of Streptococcus suis in the presence of niacin, nicotinamide, and/or anabasine.

### CROSS-REFERENCE TO RELATED APPLICATIONS

The present application claims priority to U.S. Provisional Patent Application No. 63/527,633, filed Jul. 19, 2023, the contents of which are incorporated by reference herein in its entirety.

### STATEMENT REGARDING FEDERALLY SPONSORED RESEARCH OR DEVELOPMENT

This invention was made with government support under Grant No. GM140034 awarded by the National Institutes of Health. The government has certain rights in the invention.

### SEQUENCE LISTING

The present application is being filed along with a sequence listing submitted electronically in ST.26 XML format. The Sequence Listing is provided as a file entitled “PRIN-90403 SL.xml” created on Jul. 17, 2024, and is 23,520 bytes in size. The Sequence Listing information in the ST.26 XML format is incorporated herein by reference in its entirety.

### TECHNICAL FIELD

The present disclosure is drawn to antibacterial agents, and specifically threoglucins.

### BACKGROUND

There is currently a high demand for new therapeutic antibiotics with new mechanisms of action. For example, Streptococcus suis is an opportunistic pathogen that can cause meningitis and severe systemic infection in humans. S. suis is also an important pathogen of pigs. While some modern antibiotics may be used to treat S. suis infections, conventional antibiotics that can do so have a broad spectrum of activity against many bacteria (not target-specific), and thus may also be toxic to human cells or beneficial bacteria.

### BRIEF SUMMARY

In various aspects, a composition for use in treating or preventing infections caused by Streptococcus suis may be provided. The composition may include at least one threoglucin in a carrier, the at least one threoglucin being present at a total concentration of at least 1 μM. The at least one threoglucin may include threoglucin A, threoglucin B, threoglucin C, threoglucin D, threoglucin E, threoglucin F, threoglucin G, threoglucin H, threoglucin I, threoglucin J, threoglucin K, threoglucin L, threoglucin M, threoglucin N, threoglucin O, threoglucin P, threoglucin Q, threoglucin R, or a combination thereof. In certain preferred aspects, the at least one threoglucin may include threoglucin A and/or threoglucin B, or variants thereof.

In certain aspects, each threoglucin may include a C-terminal Tryptophan (Trp)-Tyrosine (Tyr) dyad and an intermediate 1,3-oxazinane modification. Preferably, each threoglucin has no more than 6 amino acids on the C-terminal side of the intermediate 1,3-oxazinane modification and no more than 7 amino acids on the N-terminal side of the intermediate 1,3 oxazinane modification.

In certain aspects, a pharmaceutical composition may be provided. The pharmaceutical composition may include at least one threoglucin in a pharmaceutically acceptable carrier, at a total concentration of at least 1 μM. The at least one threoglucin may include threoglucin A, threoglucin B, threoglucin C, threoglucin D, threoglucin E, threoglucin F, threoglucin G, threoglucin H, threoglucin I, threoglucin J, threoglucin K, threoglucin L, threoglucin M, threoglucin N, threoglucin O, threoglucin P, threoglucin Q, threoglucin R, or a combination thereof. In certain preferred aspects, the at least one threoglucin may include threoglucin A and/or threoglucin B, or variants thereof.

In certain aspects, each threoglucin may include a C-terminal Tryptophan (Trp)-Tyrosine (Tyr) dyad and an intermediate 1,3-oxazinane modification. Preferably, each threoglucin has no more than 6 amino acids on the C-terminal side of the intermediate 1,3-oxazinane modification and no more than 7 amino acids on the N-terminal side of the intermediate 1,3 oxazinane modification.

In various aspects, a method for production of a threoglucin may be provided. The method may include growing a culture of Streptococcus suis in the presence of niacin, nicotinamide, and/or anabasine. The method may include filtering supernatant from the culture. The method may include passing the filtered supernatant through a column comprising porous graphitic carbon (PGC). The method may include generating elutions by eluting at least one threoglucin from the PGC. The method may include forming an extract by pooling and lyophilizing the elutions. The method may include purifying the extract.

### BRIEF DESCRIPTION OF DRAWINGS

FIG. 1 is a schematic illustration of a threoglucin.

FIG. 2 is a schematic illustration of one embodiment of a variant of a threoglucin.

FIG. 3 is a flowchart of a method for producing threoglucins.

FIGS. 4A-4B are growth curves for untreated (control, solid line) and threoglucin A/B-treated S. suis with concentrations ranging from 30 μM to 60 nM, broken into two graphs for simplicity—30 μM to 1 μM are shown in FIG. 4A, while 500 nM to 60 nM are shown in FIG. 4B.

FIG. 5 is a graph showing the effect of threoglucin A/B addition at OD₆₀₀ of 0.2 on the growth kinetics of S. suis.

FIG. 6 is a graph showing survival of S. suis, as measured by CFUs, after treatment of cultures with 30 μM threoglucin A/B at OD₆₀₀ of 0.2.

FIG. 7 is a graph showing survival of S. suis after treatment during exponential phase (OD₆₀₀ of 0.2) with 2 μM threoglucin A/B (+Thr.), 2 μM threoglucin A/B and 200 μM ciprofloxacin (+Thr.+Cip.), or 200 μM ciprofloxacin (+Cip.), as measured by CFU analysis. The results are scaled to growth of untreated cultures.

### DETAILED DESCRIPTION

Disclosed herein are a family of small molecules, threoglucins A-R, with novel chemical structures and potent antibiotic activity against a narrow spectrum of bacteria. This narrow spectrum of activity indicates the threoglucins could be working through a new antibiotic mechanism of action. Due to this narrow spectrum, the threoglucins could be used as highly targeted therapeutics to treat or prevent disease without disturbing the other important “beneficial” bacteria within, e.g., the human microbiome, which is a great improvement over virtually all other clinically used antibiotics.

The threoglucins are ribosomally synthesized and post-translationally modified peptides (RiPPs). The threoglucins are strongly active against Streptococcus suis, an important pathogen in livestock, notably pigs, and capable of transferring to humans.

More particularly, disclosed herein are threoglucins, small organic molecules with potent and highly selective antibiotic activity. It has been found that the threoglucins inhibit Streptococcus suis at concentrations of 1 μM. It has, however, no effect on five human cell lines tested at 60 μM.

Possible uses for the disclosed approach include deployment of the threoglucins to treat disease caused by streptococcal infections including bacterial pneumonia, bacterial meningitis, and gum disease. The disclosed approach may also be used in veterinary medicine to treat streptococcal infection in livestock, such as pigs.

The threoglucins are a new composition of matter and natural products synthesized by Streptococcus spp. Under standard growth conditions, little to no threoglucin can be detected. Methods are disclosed herein to induce the production of threoglucins and to isolate them from the original streptococcal bacterial host.

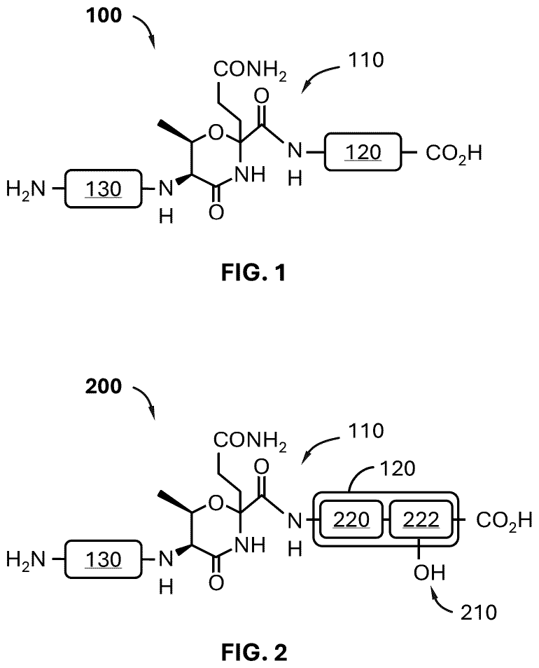

The disclosed threoglucins generally have a structure akin to that shown in FIG. 1. The threoglucin (100) may have an oxazinane heterocycle, and specifically may have an intermediate 1,3-oxazinane modification (110). This modification be formed by, e.g., an aliphatic ether linking a sidechain oxygen on a threonine with the α-carbon of an adjacent glutamine. The modification is in an intermediate location on the threoglucin. That is, there are C-terminal side amino acids (120) (i.e., amino acids on the C-terminal side of the intermediate 1,3 oxazinane modification), and N-terminal side amino acids (130) (i.e., amino acids on the N-terminal side of the intermediate 1,3 oxazinane modification).

In certain embodiments, the C-terminal side amino acids may include no more than 6 amino acids. In certain embodiments, the C-terminal side amino acids may include no more than 5 amino acids. In certain embodiments, the C-terminal side amino acids may include no more than 4 amino acids. In certain embodiments, the C-terminal side amino acids may include at least 3 amino acids. In certain embodiments, the C-terminal side amino acids may include at least 4 amino acids. In certain embodiments, the C-terminal side amino acids may include at least 5 amino acids. In certain preferred embodiments, the C-terminal side amino acids may include 6 amino acids.

In some embodiments, the threoglucin may include a C-terminal Tryptophan (Trp)-Tyrosine (Tyr) dyad (e.g., the dyad being at the C-terminal end of the C-terminal side amino acids).

In certain embodiments, the N-terminal side amino acids may include no more than 11 amino acids. In certain embodiments, the N-terminal side amino acids may include no more than 10 amino acids. In certain embodiments, the N-terminal side amino acids may include no more than 9 amino acids. In certain embodiments, the N-terminal side amino acids may include no more than 8 amino acids. In certain embodiments, the N-terminal side amino acids may include no more than 7 amino acids. In certain embodiments, the N-terminal side amino acids may include at least 4 amino acids. In certain embodiments, the N-terminal side amino acids may include at least 5 amino acids. In certain embodiments, the N-terminal side amino acids may include at least 6 amino acids. In certain preferred embodiments, the N-terminal side amino acids may include 6 or 7 amino acids.

Table 1, below, includes various threoglucins. The bolded and italicized TQ in each sequence represents the oxazinane heterocycle.

| | SEQ | Amino Acid Sequence |
|---|---|---|
| | ID | (N terminal -> |
| Name | NO: | C terminal) |
| Threoglucin A | 1 | SGCGTKR TQ QTKGWY |
| Threoglucin B | 2 | GCGTKR TQ QTKGWY |
| Threoglucin C | 3 | TLSGCGTKR TQ QTKGWY |
| Threoglucin D | 4 | LSGCGTKR TQ QTKGWY |
| Threoglucin E | 5 | SGCGTKR TQ QTKGW |
| Threoglucin F | 6 | GCGTKR TQ QTKGW |
| Threoglucin G | 7 | TLSGCGTKR TQ QTKG |
| Threoglucin H | 8 | LSGCGTKR TQ QTKG |
| Threoglucin I | 9 | SGCGTKR TQ QTKG |
| Threoglucin J | 10 | GCGTKR TQ QTKG |
| Threoglucin K | 11 | GTKR TQ QTKG |
| Threoglucin L | 12 | IETLSGCGTKR TQ QTK |
| Threoglucin M | 13 | TLSGCGTKR TQ QTK |
| Threoglucin N | 14 | LSGCGTKR TQ QTK |
| Threoglucin O | 15 | SGCGTKR TQ QTK |
| Threoglucin P | 16 | GCGTKR TQ QTK |
| Threoglucin Q | 17 | CGTKR TQ QTK |
| Threoglucin R | 18 | GTKR TQ QTK |

Variants of these threoglucins are also envisioned. As used herein, the term “variant” is used to refer to modified threoglucins that include one or more substituents, such as hydroxyl groups or methyl groups. In preferred embodiments of variants, a single substituent is introduced. For example, referring to FIG. 2, the variant (200) may contain a hydroxyl group (210) on a C-terminal amino acid (222), leaving the remaining amino acids (220) in the C-terminal side amino acids (120) unmodified. For example, four variants (oxo-Threoglucin A, oxo-Threoglucin B, oxo-Threoglucin G, and oxo-Threoglucin H) each include a hydroxyl group on the C-terminal tyrosine, but are otherwise identical to the base Threoglucin (Threoglucin A, Threoglucin B, Threoglucin G, and Threoglucin H, respectively).

Compositions containing these threoglucins or variants thereof may be useful as antibacterial compositions. For example, the composition may be useful in treating or preventing infections caused by Streptococcus suis. In some embodiments, the compositions may comprise, e.g., a pharmaceutically or veterinarily acceptable carrier.

The veterinarily acceptable carrier may provide numerous functions to the formulation, such as enhancing solubility, stability, tolerability (e.g., anti-irritant), flowability, and the like. Non-limiting examples of such veterinarily acceptable carriers include: alcohols, such as C2-C5 monoalcohols (e.g., methanol, ethanol, isopropyl (IPA), etc.), glycol ether, N-methyl pyrrolidinone (NMP), polyvinylpyrrolidinone (PVP), 2-pyrrolidone, gamma-hexylactone, methoxy propyl acetate (MPA), glycerol formal, glycerin, triacetin, d-panthenol, avenanthramides, water, or mixtures thereof.

Pharmaceutical compositions of the invention may comprise a pharmaceutically acceptable carrier. Pharmaceutically acceptable carriers may include, e.g., essentially chemically inert and nontoxic compositions that do not interfere with the effectiveness of the biological activity of the pharmaceutical composition. Non-limiting examples of such pharmaceutical carriers include: water, saline solutions, glycerol solutions, ethanol, N-(1 (2,3-dioleyloxy)propyl)N,N,N-trimethylammonium chloride (DOTMA), diolesylphosphotidyl-ethanolamine (DOPE), and liposomes.

In some embodiments, the threoglucin(s) in the composition include threoglucin A, threoglucin B, threoglucin C, threoglucin D, threoglucin E, threoglucin F, threoglucin G, threoglucin H, threoglucin I, threoglucin J, threoglucin K, threoglucin L, threoglucin M, threoglucin N, threoglucin O, threoglucin P, threoglucin Q, threoglucin R, or a combination thereof. In certain preferred embodiments, the threoglucin(s) in the composition include threoglucin A (and/or variants thereof) and/or threoglucin B (and/or variants thereof).

In certain preferred embodiments, each threoglucin includes a C-terminal Tryptophan (Trp)-Tyrosine (Tyr) dyad and an intermediate 1,3-oxazinane modification. In certain preferred embodiments, each threoglucin has no more than 6 amino acids on the C-terminal side of the intermediate 1,3-oxazinane modification and no more than 7 amino acids on the N-terminal side of the intermediate 1,3 oxazinane modification.

Such compositions should contain a therapeutically effective amount of the compound, together with a suitable amount of carrier so as to provide the form for direct administration to the patient.

In some embodiments, the total concentration of the threoglucins (or variants thereof) is at least 0.5 μM. In some embodiments, the total concentration of the threoglucins (or variants thereof) is at least 1 μM. In some embodiments, the total concentration of the threoglucins (or variants thereof) is at least 2 μM. In some embodiments, the total concentration of the threoglucins (or variants thereof) is at least 4 μM. In some embodiments, the total concentration of the threoglucins (or variants thereof) is at least 8 μM. In some embodiments, the total concentration of the threoglucins (or variants thereof) is at least 10 μM. In some embodiments, the total concentration of the threoglucins (or variants thereof) is at least 20 μM. In some embodiments, the total concentration of the threoglucins (or variants thereof) is at least 30 μM. In some embodiments, the total concentration of the threoglucins (or variants thereof) is at least 50 μM. In some embodiments, the total concentration of the threoglucins (or variants thereof) is at least 100 μM.

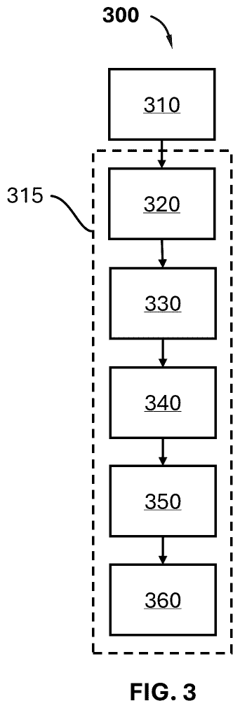

Referring to FIG. 3, a method for producing a threoglucin is shown. The method (300) may include growing (310) a culture of a Streptococcus spp., such as Streptococcus suis, in the presence of a small (under 200 g/mol) pyridine-containing molecule, and preferably a molecule such as niacin (nicotinic acid), nicotinamide, and/or anabasine. For example, the streptococcal cells may be grown in a growth medium that may be inoculated with the small pyridine-containing molecule. The concentration of the small pyridine-containing molecule in the growth medium may be, e.g., 100 μM or less, 50 μM or less, 40 μM or less, 30 UM or less, or 20 UM or less.

The species preferably includes the tqq biosynthetic gene cluster (BGC). As is known, the largest subfamily in the streptococcal RiPP network, termed the TQQ family based on a conserved amino acid motif in the precursor, encodes a 37mer peptide (TqqA), a RaS enzyme (TqqB), and a protease-transporter (TqqC). TqqB catalyzes the formation of an ether crosslink by joining the threonine sidechain oxygen to the α-carbon of the adjacent glutamine residue in TqqA.

The method may include additional steps (315) as understood in the art to isolate and purify threoglucins expressed by the cells in the presence of the small pyridine-containing molecule. The method may include filtering (320) supernatant from the culture. The method may include passing (330) the filtered supernatant through a column comprising porous graphitic carbon (PGC). The method may include generating (340) elutions by, e.g., eluting at least one threoglucin from the PGC. The method may include forming (350) an extract by pooling and lyophilizing the elutions. The method may include purifying (360) the extract.

### Example 1

S. suis was inoculated into THY media from glycerol stocks and cultured overnight at 37° C. and 5% CO₂. Overnight cultures were centrifuged (4000 g, 5 min), and the bacterial pellet resuspended in prewarmed CDMn (streptococcal chemically defined media (CDM) supplemented with 10 μM nicotinic acid). This suspension was then used to inoculate two sterile 5 L glass fermenters each with 5 L of CDMn at a starting OD 600 of 0.01. These were incubated at 37° C. for 20 h and then the cultures were centrifuged to pellet cells (15,000 g, 30 min, 4° C.). Culture supernatants were first passed through 0.2 μm filters and then run through a preconditioned open column containing ˜20 g of porous graphitic carbon (PGC). The flowthrough was discarded, and the column was washed with 500 ml of water+0.1% FA. The threoglucins were eluted from the PGC resin with 50% MeCN in water (+0.1% FA). Elutions containing threoglucin were pooled and lyophilized. The extract was then resuspended in water+0.1% FA, and threoglucins were purified by HPLC using a semi-preparative Phenomenex Omega Polar C18 column (5 μm, 10×250 mm) with a gradient from 8-25% MeCN (+0.1% FA) over 15 minutes with a flow rate of 2.5 mL/min. Threoglucin A and B were not further separated in this example, and were copurified from this method (this mixture of threoglucin A and B are referred to herein as threoglucin A/B), while other threoglucins were isolated from subsequent rounds of HPLC.

The effect of Threoglucin A/B was examined against a panel of 19 different bacteria, including S. suis and 11 other streptococci, and five human cell species.

Glycerol stocks of bacterial strains were inoculated into either lysogeny broth (LB) (B. gladioli, B. thailandensis, E. coli, S. aureus, and P. aeruginosa), or Todd-Hewitt broth supplemented with 2% yeast extract (THY) (E. faecalis, S. sanguinis, S. sobrinus, S. mutans, S. agalactiae, S. pyogenes, S. mitis, S. oralis, S. parauberis, S. porci, S. equi, and S. suis). B. gladioli, B. thailandensis, E. coli, S. aureus, and P. aeruginosa were incubated overnight at 37° C. and 200 RPM, while E. faecalis, S. sanguinis, S. sobrinus, S. mutans, S. agalactiae, S. pyogenes, S. mitis, S. oralis, S. parauberis, S. porci, S. equi, and S. suis were incubated in an atmosphere of 5% CO2 without shaking. The next morning, cultures were centrifuged at room temperature (4000 g, 5 min). Strains were resuspended in prewarmed CDMn, and then for each strain 100 μL was added to 10 mL (1% inoculum) of CDMn. 384-well plates were prepared using the 1% inoculum cultures with 50 μL of culture per well. Purified threoglucin A/B was serially diluted down the plates with a highest concentration of 30 μM. 384-well plates were then cultured at either 37° C. (B. gladioli, B. thailandensis, E. coli, S. aureus, and P. aeruginosa) or 37° C. 5% CO2 without shaking (E. faecalis, S. sanguinis, S. sobrinus, S. mutans, S. agalactiae, S. pyogenes, S. mitis, S. oralis, S. parauberis, S. porci, S. equi, and S. suis). Growth was monitored over time via OD₆₀₀ measurements using a BIOTEK® plate reader.

Threoglucin A/B exhibited an apparent half-maximal inhibitory concentration (IC₅₀) of 0.5 μM only against S. suis but did not affect any other strain tested, even at a final concentration of 30 μM. No effects were observed against five different human cell lines at 60 μM. See Table 2, below.

TABLE 2

| Organism | Threoglucin A/B IC₅₀ |
|---|---|
| Bacillus subtilis 168 | >30 μM |
| Burkholderia gladioli ATCC 10248 | >30 μM |
| Burkholderia thailandensis E264 | >30 μM |
| Enterococcus faecalis OG1RF | >30 μM |
| Escherichia coli BL21 | >30 μM |
| Pseudomonas aeruginosa PAO1 | >30 μM |
| Staphylococcus aureus Newman | >30 μM |
| Streptococcus agalactiae A909 | >30 μM |
| Streptococcus equi ATCC BAA-1716 | >30 μM |
| Streptococcus mitis CMW7705B | >30 μM |
| Streptococcus mitis NCTC 12261 | >30 μM |
| Streptococcus mutans UA159 | >30 μM |
| Streptococcus oralis E697 | >30 μM |
| Streptococcus parauberis FSL R5-303 | >30 μM |
| Streptococcus porci DSM 23759 | >30 μM |
| Streptococcus pyogenes NZ131 | >30 μM |
| Streptococcus sanguinis ATCC10556 | >30 μM |
| Streptococcus sobrinus SL1 | >30 μM |
| Streptococcus suis ATCC 43765 | 0.5 μM |
| Human Cell Line - HEK293T | >60 μM |
| Human Cell Line - HeLa | >60 μM |
| Human Cell Line - Rpe-1 | >60 μM |
| Human Cell Line - HCT116 | >60 μM |
| Human Cell Line - MDA-MB-231 | >60 μM |

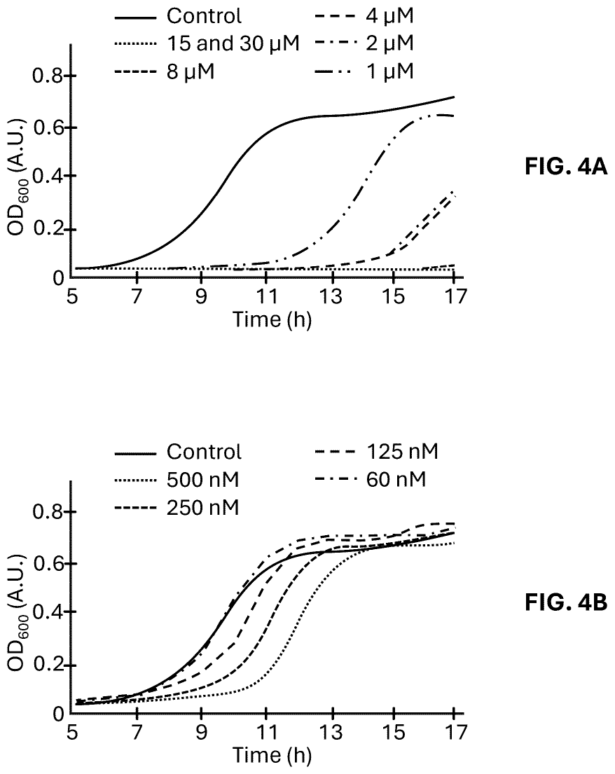

The bioactivity results suggest S. suis is the target of threoglucins. Further examination revealed concentration-dependent effects with significant growth delays at 0.5-4 μM threoglucin A/B. See FIGS. 4A-4B.

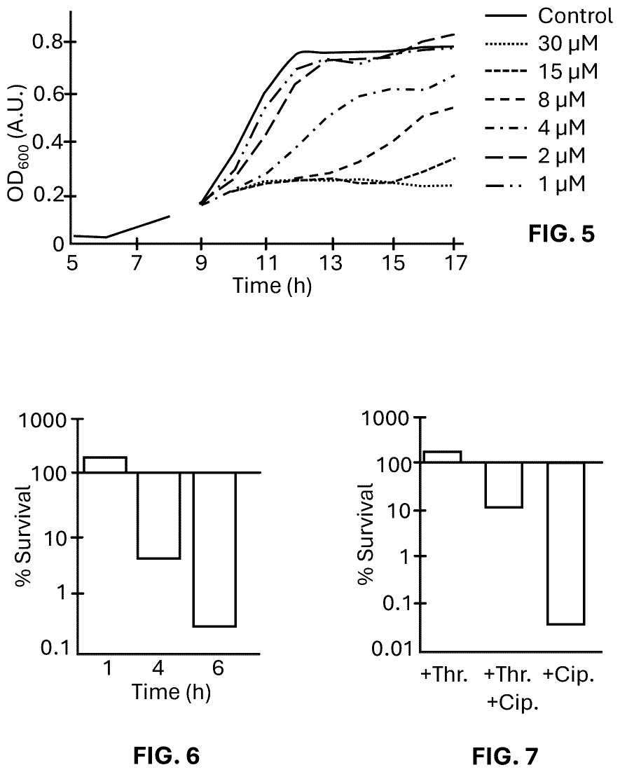

Optical densities fully recovered, suggesting the peptide is bacteriostatic at these titers. Similarly, application of threoglucin A/B at early exponential phase resulted in rapid growth arrest, from which the bacteria recovered at low doses. See FIG. 5.

Treatment with high titers resulted in loss of viability; after 6 h exposure to 30 μM threoglucin A/B, <1% of the culture remained viable based on analysis of colony forming units, suggesting the RiPP is bactericidal at these concentrations. See FIG. 6.

The recovery from low doses suggests threoglucin A/B can act as a growth-curbing or dormancy signal, which can render cells tolerant to antibiotic treatment. The ability of S. suis to survive ciprofloxacin treatment was then tested in the absence and presence of threoglucin A/B. Remarkably, the presence of the RiPP at 2 μM resulted in ˜350-fold greater viability upon treatment with 200 μM ciprofloxacin. See FIG. 7. These results suggest that threoglucin is a growth-curbing signal that can increase the tolerance of S. suis to antibiotics or other toxins, thereby providing an advantage only to the producing host, thus explaining its narrow-spectrum activity. Unnaturally high threoglucin concentrations act as selective bactericidal agents.

What is claimed:

1. A composition for use in treating or preventing infections caused by Streptococcus suis, comprising at least one threoglucin in a carrier, the at least one threoglucin being present at a total concentration of at least 1 μM.

2. The composition according to claim 1, wherein the at least one threoglucin comprises threoglucin A, threoglucin B, threoglucin C, threoglucin D, threoglucin E, threoglucin F, threoglucin G, threoglucin H, threoglucin I, threoglucin J, threoglucin K, threoglucin L, threoglucin M, threoglucin N, threoglucin O, threoglucin P, threoglucin Q, threoglucin R, or a combination thereof.

3. The composition according to claim 1, wherein the at least one threoglucin comprises threoglucin A and/or threoglucin B, or variants thereof.

4. The composition according to claim 1, wherein each threoglucin includes a C-terminal Tryptophan (Trp)-Tyrosine (Tyr) dyad and an intermediate 1,3-oxazinane modification.

5. The composition according to claim 4, wherein each threoglucin has no more than 6 amino acids on the C-terminal side of the intermediate 1,3-oxazinane modification and no more than 7 amino acids on the N-terminal side of the intermediate 1,3 oxazinane modification.

6. A pharmaceutical composition comprising at least one threoglucin in a pharmaceutically acceptable carrier, the at least one threoglucin being present at a total concentration of at least 1 μM.

7. The pharmaceutical composition according to claim 6, wherein the at least one threoglucin comprises threoglucin A, threoglucin B, threoglucin C, threoglucin D, threoglucin E, threoglucin F, threoglucin G, threoglucin H, threoglucin I, threoglucin J, threoglucin K, threoglucin L, threoglucin M, threoglucin N, threoglucin O, threoglucin P, threoglucin Q, threoglucin R, or a combination thereof.

8. The pharmaceutical composition according to claim 6, wherein the at least one threoglucin comprises threoglucin A and/or threoglucin B, or variants thereof.

9. A method for production of a threoglucin, comprising:
growing a culture of Streptococcus suis in the presence of niacin, nicotinamide, and/or anabasine.

10. The method according to claim 9, further comprising filtering supernatant from the culture.

11. The method according to claim 10, further comprising passing the filtered supernatant through a column comprising porous graphitic carbon (PGC).

12. The method according to claim 11, further comprising generating elutions by eluting at least one threoglucin from the PGC.

13. The method according to claim 12, further comprising forming an extract by pooling and lyophilizing the elutions.

14. The method according to claim 13, further comprising purifying the extract.
